(19) United States
(12) Patent Application Publication
(10) Pub. No.: US 2024/0335132 A1
(43) Pub. Date: Oct. 10, 2024

(54) SYSTEM FOR DETERMINING SPUTUM TYPE USING RESPIRATORY SOUND AND METHOD FOR DETERMINING SPUTUM TYPE

(71) Applicants: Industry-Academic Cooperation Foundation, YONSEI UNIVERSITY, Seoul (KR); THE CATHOLIC UNIVERSITY OF KOREA INDUSTRY-ACADEMIC COOPERATION FOUNDATION, Seoul (KR)

(72) Inventors: Jong Baeg KIM, Goyang-si (KR); Dae Yeon KOH, Seoul (KR); Yo Han JUNG, Seoul (KR); Hyun Jun HAN, Seoul (KR); Young Hoon JOO, Seoul (KR); Hyun Bum KIM, Seoul (KR)

(21) Appl. No.: 18/604,806

(22) Filed: Mar. 14, 2024

(30) Foreign Application Priority Data
Apr. 6, 2023 (KR) 10-2023-0045637

(51) Int. Cl. A61B 5/08; A61B 5/00

(52) U.S. Cl. CPC A61B 5/0803; A61B 5/7257; A61B 5/7267; A61B 2562/0204

(57) ABSTRACT

A system for determining a sputum type using a respiratory sound according to an embodiment may comprise a data collection unit that collects respiratory sound data from a patient who has undergone tracheostomy; an image conversion unit that receives the respiratory sound data collected by the data collection unit and converts the received respiratory sound data into a spectrogram image; and a sputum type determination unit that determines a sputum type of the patient who has undergone tracheostomy using a deep learning model based on a pattern difference between the spectrogram images converted by the image conversion unit.

### CROSS REFERENCE TO RELATED APPLICATION

The present application claims priority to Korean Patent Application No. 10-2023-0045637, filed Apr. 6, 2023, the entire contents of which is incorporated herein for all purposes by this reference.

### BACKGROUND OF THE INVENTION

### Field of the Invention

The present invention relates to a system and method for determining a sputum type using a respiratory sound, and more specifically, to a system and method for determining a sputum type using deep learning-based respiratory sound that may automatically determine whether sputum suction is necessary for a patient who has undergone tracheostomy using the respiratory sound data of the patient.

### Description of the Related Art

Recently, as the entire population is aging, the frequency of respiratory diseases such as respiratory failure, respiratory nerve damage, and carcinomas of the respiratory tract, including the larynx, is increasing. These respiratory diseases pose a risk of airway obstruction, for which tracheostomy is generally performed as a treatment method.

Tracheostomy is a surgical procedure that temporarily or permanently incises part of the trachea to secure a breathing passage. If it is necessary to maintain a breathing passage for a long time, a tracheostomy tube should be installed in the patient's trachea immediately after tracheostomy. Patients who have undergone the tracheostomy cannot easily expel sputum formed within the trachea because the tracheostomy tube does not have a ciliary function, and communication through speech is difficult, so sputum suction must be performed at the discretion of the medical staff.

When sputum accumulates in a patient's trachea or tracheostomy tube, the shape and mechanical properties of the passage through which air flows (the tracheostomy tube and the patient's trachea) change depending on the type of accumulation, resulting in differences in respiratory sounds. Since the medical staff cannot visually check the amount of sputum, they either frequently suction the patient's trachea for a certain period of time or listen to the patient's respiratory sounds before performing suction.

However, when suctioning is performed frequently for a certain period of time, the unnecessary suction generated (suctioning over time even though there is no sputum) during this process puts a burden on the patient and at the same time damages the tracheal wall or causes violent coughing. In addition, in the case of auditory-based respiratory sound diagnosis, relatively accurate judgment is possible, but there is a problem that it is difficult for medical staff or caregivers to always be near the patient and the subjective opinion of medical staff is involved.

### DOCUMENTS OF RELATED ART

(Patent Document 1) KR Registered Patent No. 10-2275936

### SUMMARY OF THE INVENTION

An object of the present invention to solve the above problems is to provide a system and method for determining a sputum type using deep learning-based respiratory sound that can automatically determine whether sputum suction is necessary for a patient who has undergone tracheostomy using the respiratory sound data of the patient.

The technical objects to be achieved by the present invention are not limited to the technical objects mentioned above, and other technical objects not mentioned may be clearly understood by those skilled in the art from the following descriptions.

In order to achieve the above technical object, a system for determining a sputum type using a respiratory sound according to an embodiment of the present invention may comprise a data collection unit that collects respiratory sound data from a patient who has undergone tracheostomy; an image conversion unit that receives the respiratory sound data collected by the data collection unit and converts the received respiratory sound data into a spectrogram image; and a sputum type determination unit that determines a sputum type of the patient who has undergone tracheostomy using a deep learning model based on a pattern difference between the spectrogram images converted by the image conversion unit.

In an embodiment of the present invention, the respiratory sound data collected by the data collection unit may be classified into respiratory sound data requiring sputum suction and normal respiratory sound data.

In an embodiment of the present invention, the deep learning model may classify the sputum type of the patient into a first sputum type that shows continuous and extensive sound pressure in a frequency range of 2 kHz or more on the spectrogram image, a second sputum type that shows repetitive vertical lines due to low-frequency vibration, and a normal type showing a relative small sound pressure.

In an embodiment of the present invention, the system may further comprise a respiratory sound extraction unit using the respiratory sound, wherein the respiratory sound extraction unit may extract respiratory sound sample data corresponding to one breathing cycle from the respiratory sound data collected by the data collection unit.

In an embodiment of the present invention, the image conversion unit may convert the respiratory sound data into a spectrogram image through short-time Fourier transform (STFT).

In an embodiment of the present invention, the deep learning model may be implemented as a convolution neural network (CNN).

In an embodiment of the present invention, the system may further comprise a model verification unit, wherein the model verification unit may verify an accuracy of the deep learning model by using already classified respiratory sound data as input data.

In an embodiment of the present invention, the model verification unit may verify an accuracy of the deep learning model using a predetermined performance evaluation index.

A method for determining a sputum type using a respiratory sound according to an embodiment of the present invention may comprise a collection step of collecting respiratory sound data from a patient who has undergone tracheostomy; a conversion step of converting the collected respiratory sound data into a spectrogram image; and a determination step of determining a sputum type of the patient who has undergone tracheostomy using a deep learning model based on a pattern difference between the converted spectrogram images.

In an embodiment of the present invention, the method may further comprise a verification step of verifying an accuracy of the deep learning model by using already classified respiratory sound data as input data.

### BRIEF DESCRIPTION OF THE DRAWINGS

FIG. 1 is a block diagram schematically showing a configuration of a system for determining a sputum type using a respiratory sound according to an embodiment of the present invention.

FIG. 2 is a flowchart schematically showing an operation sequence of a method for determining a sputum type using a respiratory sound according to an embodiment of the present invention.

FIG. 3 is a flowchart showing an operation of converting respiratory sound data into a spectrogram image in an image conversion unit of a system for determining a sputum type using a respiratory sound according to an embodiment of the present invention.

FIG. 4 is a diagram showing a waveform of a respiratory sound sample according to a sputum type and a converted spectrogram image in a system for determining a sputum type using a respiratory sound according to an embodiment of the present invention.

FIG. 5 is an example diagram showing (a) a confusion matrix and (b) a classification performance evaluation index for verification of a deep learning model of a system for determining a sputum type using a respiratory sound according to an embodiment of the present invention.

FIG. 6 is a graph showing a receiver operating characteristics (ROC) curve in case where a system for determination a sputum type using a respiratory sound according to an embodiment of the present invention classifies the sputum type into two.

### DETAILED DESCRIPTION OF THE INVENTION

Hereinafter, the present invention will be explained with reference to the accompanying drawings. The present invention, however, may be modified in various different ways, and should not be construed as limited to the embodiments set forth herein. Also, in order to clearly explain the present invention, portions that are not related to the present invention are omitted, and like reference numerals are used to refer to like elements throughout.

Throughout the specification, it will be understood that when an element is referred to as being “connected (accessed, contacted, coupled) to” another element, this includes not only cases where the elements are “directly connected,” but also cases where the elements are “indirectly connected” with another member therebetween. Also, it will also be understood that when a component “includes” an element, unless stated otherwise, this does not mean that other elements are excluded, but that other element may be further added.

The terms used herein are only used to describe specific embodiments and are not intended to limit the present invention. The singular forms are intended to include the plural forms as well, unless the context clearly indicates otherwise. In the specification, it will be further understood that the terms “comprise” and “include” specify the presence of stated features, integers, steps, operations, elements, components, and/or combinations thereof, but do not preclude in advance the possibility of the presence or addition of one or more other features, integers, steps, operations, elements, components, and/or combinations.

Hereinafter, the embodiments of the present invention will be explained with reference to the accompanying drawings.

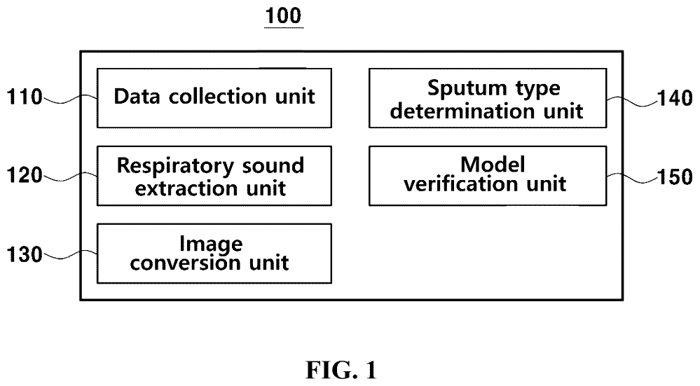

FIG. 1 is a block diagram schematically showing a configuration of a system for determining a sputum type using a respiratory sound according to an embodiment of the present invention.

Referring to FIG. 1, a sputum type determination system 100 may be configured to include a data collection unit 110, a respiratory sound extraction unit 120, an image conversion unit 130, a sputum type determination unit 140, and a model verification unit 150.

The data collection unit 110 may be provided to collect respiratory sound data, and the respiratory sound data may be classified into, for example, respiratory sound data of a patient who has undergone tracheostomy and normal respiratory sound data.

The respiratory sound of a patient who has undergone tracheostomy may be collected by installing a microphone at a location approximately 2 to 3 cm away from the breathing passage outlet of a tracheostomy tube in order to fully identify a difference in sounds depending on the type of sputum accumulation in the patient who has undergone tracheostomy. The respiratory sound data collected over a long period of time with the microphone may be classified into the following three types: a respiratory sound showing a high-frequency sound due to narrowing of the breathing passage among the respiratory sounds determined by a plurality of otolaryngologists to require sputum suction (sputum type 1), a respiratory sound showing the sound of sputum vibrating among the respiratory sounds to require sputum suction (sputum type 2), and a normal respiratory sound requiring no sputum suction (normal type), so that the respiratory sound data may be formed. The data collection unit 110 may be provided to collect the data. The types of respiratory sounds are not limited to the three types and may be classified into two types: a sputum type and a normal type.

The respiratory sound extraction unit 120 may be configured to extract respiratory sound sample data corresponding to one breathing cycle from the respiratory sound data collected by the data collection unit 110. The respiratory sound sample data may be configured to be extracted only for respiratory sound data section in which the classification results of the plurality of otolaryngologists match.

The image conversion unit 130 may be provided to receive the respiratory sound data collected by the data collection unit 110 and convert the respiratory sound data into a spectrogram image through short-time Fourier transform (STFT).

Specifically, the image conversion unit 130 may be configured to divide the respiratory sound sample data extracted by the respiratory sound extraction unit 120 into a plurality of Hamming windows, perform discrete Fourier transform (DFT) on the data divided by the plurality of Hamming windows, and generate a spectrogram image by merging the discrete Fourier transformed data in time order.

The sputum type determination unit 140 may be configured to determine the sputum type of the patient who has undergone tracheostomy using a deep learning model based on a pattern difference between the spectrogram images converted by the image conversion unit 130. The deep learning model may be implemented as a convolution neural network (CNN), and may include, for example, at least one selected from the group consisting of AlexNet, VGGNet, ResNet, Inception, and MobileNet.

The deep learning model may classify the sputum type of the patient into a first sputum type that shows continuous and extensive sound pressure in the frequency range of 2 kHz or more on the spectrogram image, a second sputum type that shows repetitive vertical lines due to low-frequency vibration, and a normal type showing a relative small sound pressure, but is not limited thereto.

The model verification unit 150 may be configured to verify the accuracy of the deep learning model by using the already classified respiratory sound data as input data. The model verification unit 150 may also be configured to verify the accuracy of the deep learning model using a predetermined performance evaluation index. In case where the sputum type is classified into the first sputum type, the second sputum type, and the normal type, the performance evaluation index of the model verification unit 150 may be configured to include an overall accuracy, which represents a rate of correctly classified data among all respiratory sound data, and a sputum classification accuracy, which represents a rate of correctly classified data when the respiratory sounds that actually require suction are classified as the respiratory sounds that require suction.

In addition to the above case, in case where the sputum type is classified into two types, the first and second sputum types and the normal type, the performance evaluation index may be configured to include a sensitivity, which represents a rate of data classified as respiratory sounds requiring suction among respiratory sounds that actually require suction, a specificity, which represents a rate of data classified as normal respiratory sounds among actual normal respiratory sounds, and an area under curve (AUC), which represents an area of the region under a receiver operating characteristic curve.

The convolutional neural network is a neural network that learns the optimal kernel combination by repeating convolution calculations between two-dimensional data such as images and kernels, and is a neural network that learns common features between images without separate manual feature extraction. The convolutional neural network may create accurate pattern recognition or image classification training models, and is widely used in the field of imagable biosignal classification.

In the present invention, for example, the spectrogram imaged respiratory sound samples for each of the first sputum type, the second sputum type, and the normal type may be arbitrarily separated into a training data set and verification data, in equal rate, preferably 7:3 or 8:2. The training data and verification data separated by the type may constitute an entire training data set and a verification data set, respectively. The entire training data set is trained through the convolutional neural network, and common features of images for each type can be learned. The deep learning model, which is implemented as the convolutional neural network model trained in this way, may be provided so that the model verification unit 150 inputs a verification data set, compares predicted results with actual results, evaluates a classification performance, and selects an optimal model.

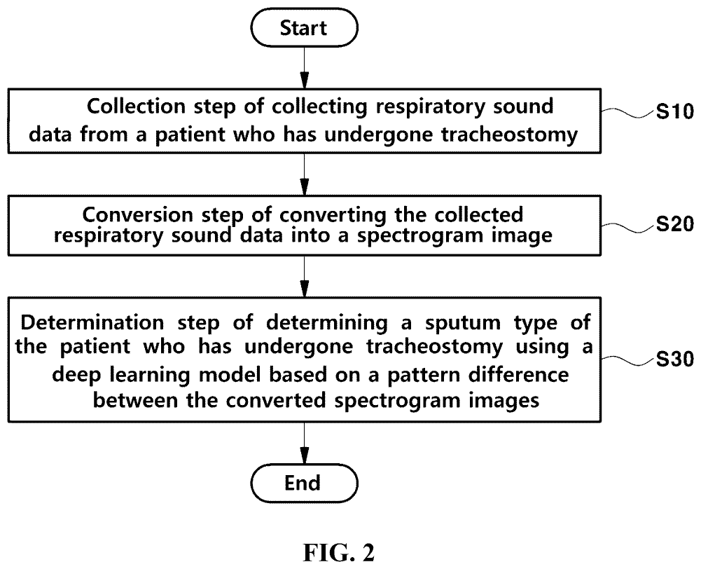

FIG. 2 is a flowchart schematically showing an operation sequence of a method for determining a sputum type using a respiratory sound according to an embodiment of the present invention.

Referring to FIG. 2, a method for determining a sputum type using a respiratory sound may be configured to include a collection step of collecting respiratory sound data from a patient who has undergone tracheostomy (S10); a conversion step of converting the collected respiratory sound data into a spectrogram image (S20); and a determination step of determining a sputum type of the patient who has undergone tracheostomy using a deep learning model based on a pattern difference between the converted spectrogram images (S30).

The method for determining a sputum type using a respiratory sound may further include a verification step of verifying the accuracy of the deep learning model by using the already classified respiratory sound data as input data.

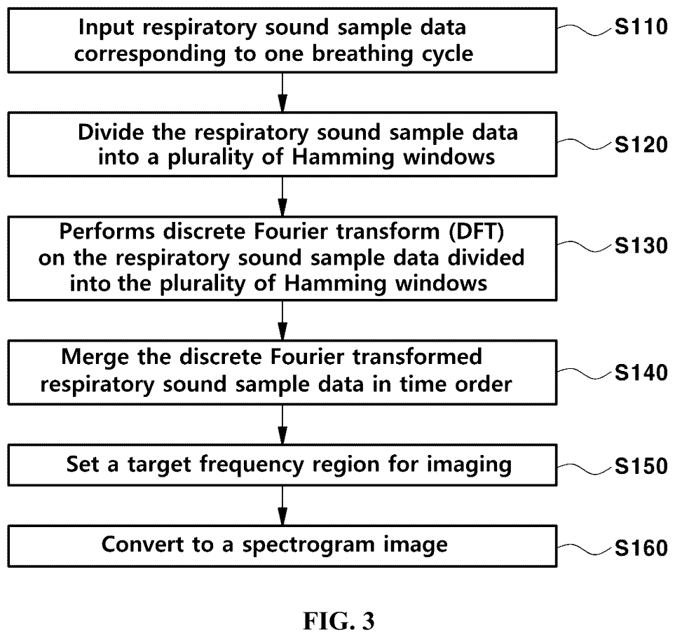

FIG. 3 is a flowchart showing an operation of converting respiratory sound data into a spectrogram image in an image conversion unit of a system for determining a sputum type using a respiratory sound according to an embodiment of the present invention.

Referring to FIG. 3, the operation of the image conversion unit 130 inputs the respiratory sound sample data corresponding to one breathing cycle (S110) and divides the respiratory sound sample data into a plurality of Hamming windows (S120), and performs discrete Fourier transform (DFT) on the respiratory sound sample data divided into the plurality of Hamming windows. The discrete Fourier transformed respiratory sound sample data are merged in time order (S140), a target frequency region for imaging is set (S150), and converted to a spectrogram image (S160).

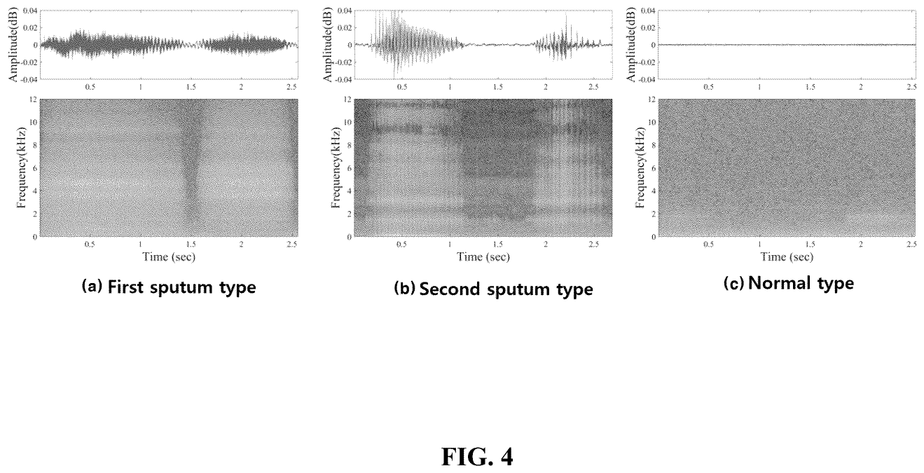

For example, one respiratory sound sample data may be divided into 512 Hamming windows, and the sample data corresponding to one Hamming window may be discrete Fourier transformed into 512 points. The Hamming window data converted to a frequency spectrum are merged in time order, and the frequency range of 0 to 12 kHz, where changes in breathing are clearly visible in the merged continuous spectrum data, may be set as a target frequency range. In this case, the number of pixels of the spectrogram image may be set to 640×360 pixels as shown in FIG. 4. Here, the number of Hamming windows, the number of points applied to the discrete Fourier transform, the frequency range, and the number of pixels in the image are not limited thereto, and in addition, conversion to a spectrogram image with a 1/3 octave scale or a melody frequency (mel-frequency) scale rather than a linear frequency range may be applied.

FIG. 4 is a diagram showing a waveform of a respiratory sound sample according to a sputum type and a converted spectrogram image in a system for determining a sputum type using a respiratory sound according to an embodiment of the present invention.

Referring to FIG. 4, the sputum type may be classified into (a) the first sputum type, which is a case in which high-intensity sputum is fixed in the breathing passage and narrows the cross-sectional area of the passage, producing continuous, wide, and high sound pressure respiratory sounds in the frequency range of 2 kHz or higher, (b) the second sputum type, which is a situation where deformable sputum is attached to a specific location in the trachea or a tracheostomy tube, causing a gurgling respiratory sound due to low-frequency vibration of the deformable sputum, causing repeated vertical lines to appear on the spectrogram image, and (c) the normal type, which is a case in which there are almost no obstacles in the breathing passage and the respiratory sounds of relatively low sound pressure are observed in the frequency range below 2 kHz.

According to the above classification, (a), (b), and (c) may be clearly distinguished visually, indicating that it is possible to determine the sputum type in the patient who has undergone tracheostomy using the spectrogram image.

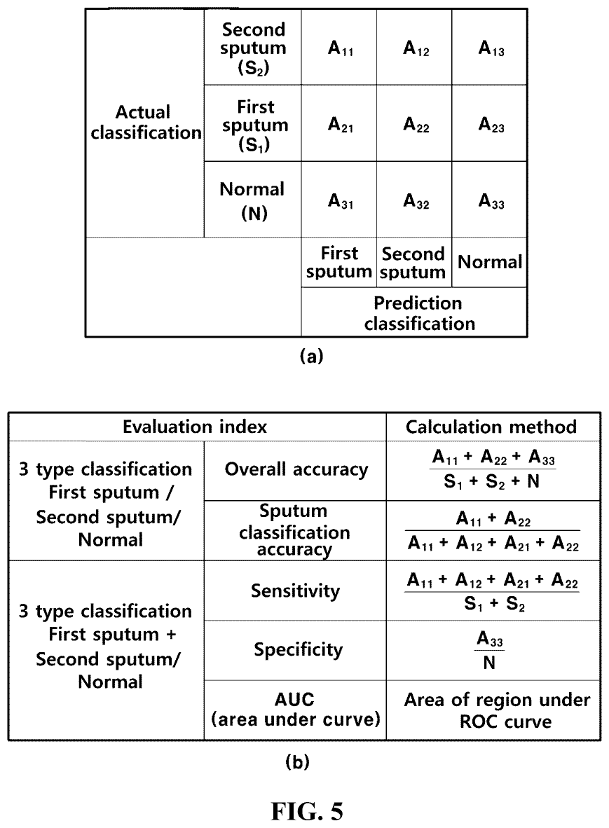

FIG. 5 is an example diagram showing (a) a confusion matrix and (b) a classification performance evaluation index for verification of a deep learning model of a system for determining a sputum type using a respiratory sound according to an embodiment of the present invention.

Referring to FIG. 5, the model verification unit 150 may verify the accuracy of the deep learning model based on (a) the confusion matrix and (b) the classification performance evaluation index in FIG. 5.

In case of classifying the respiratory sounds into 3 types: the first sputum type/the second sputum type/the normal type, the evaluation index for the classification performance of the respiratory sounds of the patient who has undergone tracheostomy according to the deep learning model is 5 as in (b), depending on the case of classification into two types: respiratory sounds requiring suction (first sputum+second sputum)/normal. FIG. 5 is only an example, and it is also possible to reflect other performance evaluation indicators.

TABLE 1

| | | | Sputum |
|---|---|---|---|
| 3 type | | Overall | classification |
| classification | Classifier name | accuracy | accuracy |
| Spectrogram | AlexNet | 0.8938 | 0.9660 |
| Convolutional | VGG_16 | 0.8799 | 0.9509 |
| neural network | ResNet_50 | 0.9027 | 0.9489 |
| | Inception_v3 | 0.9001 | 0.9461 |
| | MobileNet | 0.8951 | 0.9514 |

Table 1 above shows the overall accuracy and sputum classification accuracy calculated as the performance evaluation index for each case when AlexNet, VGGNet, ResNet, Inception, and MobileNetof the convolutional neural networks were used as the deep learning model in the three type classification in FIG. 5.

Considering Table 1 above, ResNet shows the highest performance on the overall accuracy evaluation index at 0.9027, and AlexNet shows the highest performance in terms of sputum classification accuracy. According to the above evaluation index, for all convolutional neural network models, the overall accuracy has the performance of at least 0.8799 and the sputum classification accuracy has the performance of at least 0.9461.

TABLE 2

| 2 type | | | | |
|---|---|---|---|---|
| classification | Classifier name | Sensitivity | Specificity | AUC |
| Spectrogram | AlexNet | 0.9401 | 0.8690 | 0.9586 |
| Convolutional | VGG_16 | 0.9341 | 0.8655 | 0.9527 |
| neural network | ResNet_50 | 0.9381 | 0.9241 | 0.9650 |
| | Inception_v3 | 0.9261 | 0.9414 | 0.9608 |
| | MobileNet | 0.9441 | 0.8897 | 0.9549 |

Table 2 above shows sensitivity, specificity, area under curve (AUC) calculated as the performance evaluation index for each case when AlexNet, VGGNet, ResNet, Inception, and MobileNet of the convolutional neural networks are used as the deep learning model in the two type classification in FIG. 5.

Considering Table 2 above, all convolutional neural network models achieved the performance index of the sensitivity of 0.9261, the specificity of 0.8655, and the AUC of 0.9527 or higher. From a patient care perspective, the higher the sensitivity, the better the model can prevent a patient's emergency situation. Considering that MobileNet achieved the highest sensitivity of 0.9441 when applied, MobileNet is the model with the best performance from a patient care perspective.

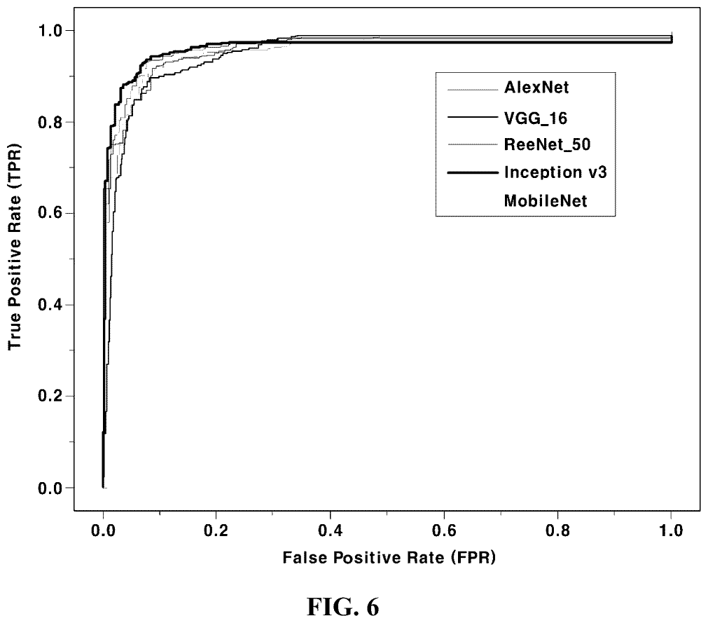

FIG. 6 is a graph showing a receiver operating characteristics (ROC) curve in case where a system for determination a sputum type using a respiratory sound according to an embodiment of the present invention classifies the sputum type into two.

The receiver operating characteristics curve refers to a curve drawn with sensitivity and specificity. ROC analysis is mainly used to determine the usefulness of a test tool or to evaluate the accuracy of a test, and may also be used to set a cut point for the test in the development of a tool for diagnosis. For this analysis, there must be at least two variables, and one of the two variables must be a dichotomous variable indicating a diagnosis or test result.

Referring to FIG. 6, the x-axis, false positive rate (FPR), represents a rate of a case where the diagnosis is negative (normal) when positive is predicted (a condition requiring sputum suction). The y-axis, true positive rate (TPR), represents a rate of a case where the diagnosis matches positive when a positive is predicted. In FIG. 6, the area of the region under the receiver operating characteristics curve represents AUC, and as shown in Table 2, ResNet shows the highest value of 0.9650, and VGG shows the lowest value of 0.9527. Therefore, according to the receiver operating characteristics (ROC) curve, it may be determined that ResNet shows the best performance.

The deep learning model shows differences in each evaluation performance index depending on the convolutional neural network model used. In this respect, the convolutional neural network model may be differently selected depending on which performance index is weighted among the evaluation performance indexes.

The system and method for determining a sputum type using a respiratory sounds described above may be implemented with hardware components, software components, and/or a combination of hardware components and software components. For example, the system, the device, the method, and the components described in the embodiments may be implemented using one or more general-purpose computers or special-purpose computers such as a processor, a controller, a central processing unit (CPU), a graphics processing unit (GPU), an arithmetic logic unit (ALU), a digital signal processor, a microcomputer, a field programmable gate array (FPGA), a programmable logic unit (PLU), a microprocessor, application specific integrated circuits (ASICS), a server, or any other device capable of executing and responding to instructions.

The above description of the present invention is used for illustration and those skilled in the art will understand that the present invention can be easily modified to other detailed forms without changing the technical spirit or an essential feature thereof. Therefore, the aforementioned exemplary embodiments are all illustrative in all aspects and are not limited. For example, each component described as a single type may be implemented to be distributed and similarly, components described to be distributed may also be implemented in a combined form.

The scope of the invention is to be defined by the scope of claims provided below, and all variations or modifications that can be derived from the meaning and scope of the claims as well as their equivalents are to be interpreted as being encompassed within the scope of the present invention.

According to an embodiment of the present invention, a system and method for determining a sputum type using deep learning-based respiratory sound that automatically determines whether sputum suction is necessary for a patient who has undergone tracheostomy using the respiratory sound data of the patient may be provided, and objective classification results for the sputum type may be provided to medical staff and caregivers and an efficient patient care guide may be presented.

The effects of the present invention are not limited to the above-mentioned effects, and it should be understood that the effects of the present invention include all effects that could be inferred from the configuration of the invention described in the detailed description of the invention or the appended claims.

### DESCRIPTION OF REFERENCE NUMERALS

100: a sputum type determination system 100
110: a data collection unit
120: a respiratory sound extraction unit
130: an image conversion unit
140: a sputum type determination unit 140
150: a model verification unit 150.

What is claimed is:

1. A system for determining a sputum type using a respiratory sound, the system comprising at least one processor configured to:
collect respiratory sound data from a patient who has undergone tracheostomy;
receive the respiratory sound data collected by the at least one processor and convert the received respiratory sound data into a spectrogram image; and
determine a sputum type of the patient who has undergone tracheostomy using a deep learning model based on a pattern difference between spectrogram images converted by the at least one processor.

2. The system of claim 1, wherein the respiratory sound data collected by the at least one processor is classified into respiratory sound data requiring sputum suction and normal respiratory sound data.

3. The system of claim 1, wherein the deep learning model is configured to classify the sputum type of the patient into a first sputum type that shows continuous and extensive sound pressure in a frequency range of 2 kHz or more on the spectrogram image, a second sputum type that shows repetitive vertical lines due to low-frequency vibration, and a normal type showing a relative small sound pressure.

4. The system of claim 1, wherein the at least one processor is configured to extract respiratory sound sample data corresponding to one breathing cycle from the respiratory sound data collected by the at least one processor.

5. The system of claim 4, wherein the at least one processor is configured to convert the respiratory sound data into a spectrogram image through short-time Fourier transform (STFT).

6. The system of claim 1, wherein the deep learning model is implemented as a convolution neural network (CNN).

7. The system of claim 1, wherein the at least one processor is configured to verify an accuracy of the deep learning model by using already classified respiratory sound data as input data.

8. The system of claim 7, wherein the at least one processor is configured to verify an accuracy of the deep learning model using a predetermined performance evaluation index.

9. A method for determining a sputum type using a respiratory sound, comprising:
collecting respiratory sound data from a patient who has undergone tracheostomy;
converting the collected respiratory sound data into a spectrogram image; and
determining a sputum type of the patient who has undergone tracheostomy using a deep learning model based on a pattern difference between converted spectrogram images.

10. The method of claim 9, further comprising verifying an accuracy of the deep learning model by using already classified respiratory sound data as input data.
